(19) United States
(12) Patent Application Publication
(10) Pub. No.: US 2008/0318869 A1
(43) Pub. Date: Dec. 25, 2008

(54) Method to Promote Wound Healing

(75) Inventor: Mark L. Witten, Tucson, AZ (US)

Correspondence Address: JONES DAY, 222 EAST 41ST ST, NEW YORK, NY, 10017, US

(73) Assignee: IMMUNEREGEN BIOSCIENCES, INC., Scottsdale, AZ (US)

(21) Appl. No.: 11/666,474

(22) PCT Filed: Oct. 25, 2005
(86) PCT No.: PCT/US05/38646

§ 371 Date: Apr. 14, 2008

Related U.S. Application Data
(60) Provisional application No. 60/622,015, filed on Oct. 27, 2004.

(51) Int. Cl. A61K 38/08; A61P 17/02

(52) U.S. Cl. 514/15

(57) ABSTRACT

Wound healing is impaired in irradiated animals. Substance P and its active analogs promote wound healing in irradiated animals. Substance P and its analogs can be used to treat animals irradiated by a nuclear weapon, a nuclear accident, or a therapeutic regimen, for example. Wounds which are susceptible to such treatment include surgical wounds, weapons wounds, disease-caused wounds, bed sores, diabetic ulcers, etc.

### FIELD OF THE INVENTION

The invention relates to the field of wound healing. In particular, it relates to treatment of wounds inflicted by disease, surgery, injury, etc. More particularly it relates to treatment of wounds in irradiated individuals.

### BACKGROUND OF THE INVENTION

Combined radiation and wound injury occurs after severe nuclear accidents that accompany explosions or nuclear attacks. High doses of ionizing radiation can cause bone marrow aplasia and delay wound healing. Shi et al. Radiat Res. 2004 Jul;162(l):56-63.

The wound healing process is adversely effected by irradiation. Irradiation causes a delay in the overall process. The early phase inflammatory response is inhibited. The formation and maturation of granulation tissue is retarded. And the reepithelialization process is delayed. These result in an overall prolongation of healing time Gu et al., J Environ Pathol Toxicol Oncol. 1998; 17:117-23. Particular components of the wound healing process that are affected include infiltrating macrophages and neutrophils, blood vessels, fibroblasts, collagen synthesis and secretion. Ibid.

There is a continuing need in the art for remedies and tools to help ameliorate the after-effects of nuclear accidents or attacks. There is a continuing need in the art for remedies and tools to help ameliorate the side-effects of therapeutic radiation.

### BRIEF SUMMARY OF THE INVENTION

According to the present invention a method is provided for stimulating wound healing in a radiation-exposed mammal. An effective amount of Substance P or an analog thereof is administered to a mammal that has been exposed to radiation and that has a wound. The analog is selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸ ]-substance P, [p-Cl-Phe^{7,8}]-substance P, [Sar⁹,Met (O₂)¹¹]-substance P, and analogs having the amino acid backbone RPKPQQFFGLM-NH₂. Healing of the wound is stimulated.

### BRIEF DESCRIPTION OF THE DRAWINGS

FIG. 1 shows the tail with bite wounds of a mouse that was subjected to irradiation.

FIG. 2 shows the tail that had bite wounds of a mouse that was subjected to irradiation and treatment with [Sar⁹,Met (O₂)¹¹]-substance P.

### DETAILED DESCRIPTION OF THE INVENTION

Animals with wounds that have been irradiated do not heal as well as non-irradiated animals. It is a discovery of the present invention that substance P or its bioactive analogs can ameliorate the negative effects of irradiation on -the wound healing process.

Substance P (RPKPQQFFGLM-NH₂; SEQ ID NO: 1) or a bioactive analog thereof such as Sar⁹,Met(O₂)¹¹-Substance P can be administered to stimulate wound healing. The bioactive analog can be selected from the group consisting of [Met-OH¹¹]-substance P, [Met-OMe¹¹]-substance P, [Nle¹¹]-substance P, [Pro⁹]-substance P, [Sar⁹]-substance P, [Tyr⁸]-substance P, Sar⁹, Met(O₂)¹¹-Substance P, and [p-Cl-Phe^{7,8}]-substance P. Other compounds which function in the same way can be identified by their ability to compete with substance P for binding to its receptor (NK-1) or for their ability to agonize the NK-1 receptor. Compounds which have the same amino acid backbone as substance P can be routinely modified and tested for receptor agonist activity. Routine assays for such activities are known in the art and can be used.

The substance P or analog can be administered by any method known in the art, including via aerosol inhalation. Intravenous, topical, intratracheal, intrabronchial, intramuscular, sublingual, and oral administrations can also be used. Suitable dosages include 0.05 to 5 nanomolar substance P or analog for administration, or 0.1 to 2 nanomolar, or 0.5 to 1.5 nanomolar. For aerosol administration dosages include 0.05 to 5 micromolar substance P or analog, preferably 0.1 to 2 micromolar, and more preferably 0.5 to 1.5 micromolar. For direct intramuscular injection a 2 micromolar solution can be used, for example. Other useful concentration ranges of substance P or its bioactive analog in an aerosol administered is between 0.001 and 75 μM. It can be advantageously administered as a liquid at a concentration between about 0.1 and 10 μM. Concentrations for topical administration are in the range of 1μM to 50 μM . Amounts to be administered are typically between 1 μM and 10 μM.

Wounds which are amenable to treatment according to the present invention are those on the surface as well as internal to an animal body. The wounds may be caused by accident, disease, or purposefully. The wounds can, for example, be surgical wounds. Amenable wounds include but are not limited to cutaneous wounds, muscular wounds, osseus lesions, gastrointestinal anastamoses, decubitus ulcers, gastrointestinal ulcers, and burn wounds. The methods of the present invention can be applied to any mammal, including humans, horses, sheep, primates such as monkeys, apes, gibbons, chimpanzees, rodents such as mice, rats, guinea pigs, hamsters, ungulates such as cows.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### EXAMPLES

### Example 1

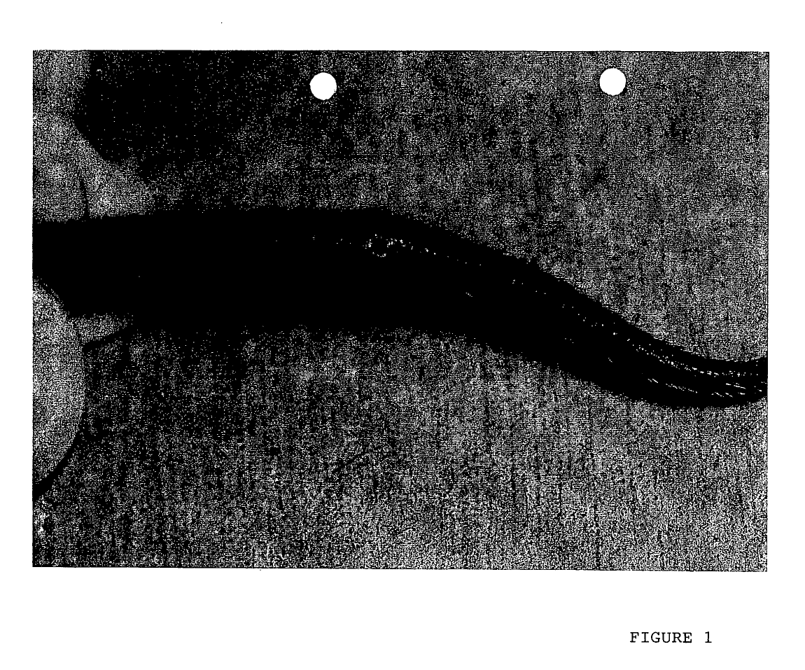

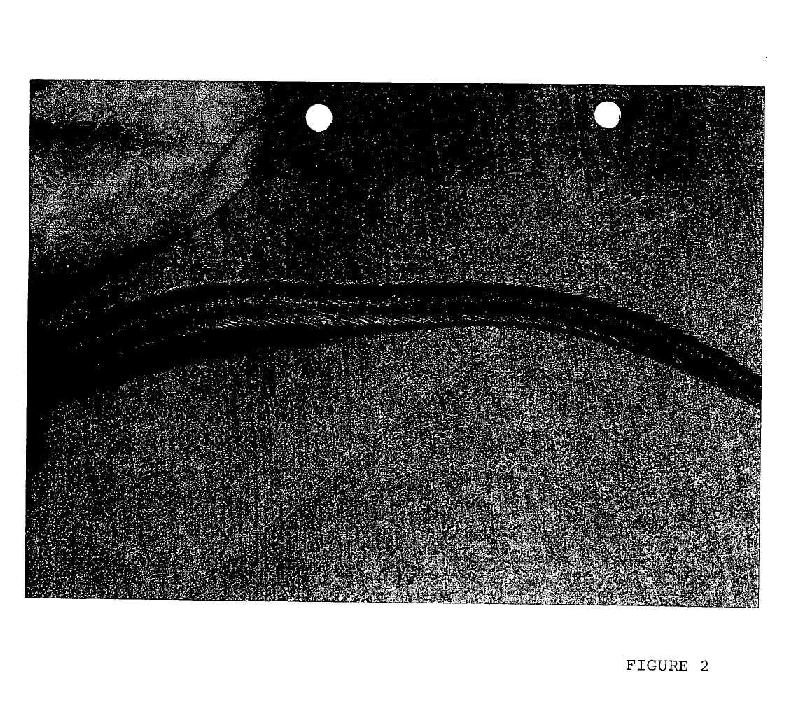

The male mice fought while confined with four mice/bin. They sustained extensive tail wounds from biting. We then exposed the mice to either 7, 8, or 9 Gy 60 Cobalt gamma radiation in a single acute dose. [Sar⁹,Met(0₂)¹¹]-substance P was administered by direct muscle injection in a 0.5 ml bolus at 2 micromolar concentration. Control animals received injections of normal saline. At 7 weeks post-radiation exposure, the control mice had unhealed tail wounds (see FIG. 1) while the [Sar⁹,Met(O₂)¹¹]-substance P-treated mice did not have any unhealed tail wounds (see FIG. 2).

I claim:

1. A method of stimulating wound healing in a radiation-exposed mammal, comprising:
administering an effective amount of Substance P or an analog thereof to a mammal that has been exposed to radiation and that has a wound, wherein said analog is selected from the group consisting of [Met-OH¹¹ ]-substance P, [Met-OMe¹¹]-substance P, [Nle¹¹]-substance P, [Pro⁹]-substance P, [Sar ⁹]-substance P, [Tyr⁸ ]-substance P, [p-Cl-Phe^{7,8}]-substance P, [Sar⁹,Met (O₂)¹¹]-substance P, and analogs having the amino acid backbone RPKPQQFFGLM-NH₂, whereby healing of the wound is stimulated.

2. The method of claim 1 wherein Substance P is administered.

3 . The method of claim 1 wherein [Met-OH¹¹]-Substance P is administered.

4. The method of claim 1 wherein [Met-OMe¹¹]-Substance P is administered.

5. The method of claim 1 wherein [Nle¹¹]-Substance P is administered.

6. The method of claim 1 wherein [Pro⁹]-Substance P is administered.

7. The method of claim 1 wherein [Sar⁹]-Substance P is administered.

8. The method of claim 1 wherein [Tyr⁸]-Substance P is administered.

9. The method of claim 1 wherein [p-Cl-Phe^{7,8}]-Substance P is administered.

10. The method of claim 1 wherein [Sar⁹,Met (O₂)¹¹]-Substance P is administered.

11. The method of claim 1 wherein the step of administering is performed by injection into a muscle.

12. The method of claim 1 wherein the step of administering is performed by topical administration.

13. The method of claim 1 wherein the step of administering is performed by intravenous-administration.

14. The method of claim 1 wherein the step of administering is performed by administration of an aerosol to the nose.

15. The method of claim 1 wherein the step of administering is performed by administration of an aerosol to the lungs.

16. The method of claim 1 wherein the mammal is a human.

17. The method of claim 1 wherein the mammal is a rodent.

18. The method of claim 1 wherein the mammal is a bovid.

19. The method of claim 1 wherein the mammal is a dog.

20. The method of claim 1 wherein the mammal is a cat.

21. The method of claim 1 wherein the mammal was exposed to fall-out from a nuclear explosion.

22. The method of claim 1 wherein the mammal was exposed to therapeutic radiation.

23. The method of claim 1 wherein the wound is a surgical wound.

24. The method of claim 1 wherein the wound is a pressure wound.

25. The method of claim 1 wherein the mammal was exposed to ionizing radiation.

26. The method of claim 1 wherein the mammal was exposed to gamma radiation.

27. The method of claim 1 wherein the mammal was occupationally exposed to radiation.

28. The method of claim 1 wherein the wound is cutaneous.

29. The method of claim 1 wherein the wound is muscular.

30. The method of claim 1 wherein the wound is an osseus lesion.

31. The method of claim 1 wherein the wound is a gastrointestinal anastamosis.

32. The method of claim 1 wherein the wound is a burn wound.

33. The method of claim 1 wherein the wound is a decubitus ulcer.


SEQUENCE LISTING (1 sequence)

SEQ ID NO: 1 - protein, 11 aa, Homo sapiens
  MOD_RES (11)...(11)
RPKPQQFFGLM